(19) United States
(12) Patent Application Publication
(10) Pub. No.: US 2008/0138820 A1
(43) Pub. Date: Jun. 12, 2008

(54) METHOD FOR DETERMINING GENOTOXICITY

(75) Inventor: NICHOLAS THOMAS, CARDIFF (GB)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE UK LIMITED, LITTLE CHALFONT (GB)

(21) Appl. No.: 11/950,591

(22) Filed: Dec. 5, 2007

(30) Foreign Application Priority Data
Dec. 7, 2006 (GB) 0624462.8

(51) Int. Cl. C12Q 1/68; C12N 15/87; C12N 5/06

(52) U.S. Cl. 435/6; 435/325; 435/455

(57) ABSTRACT

Disclosed are compositions and methods which provide an integrated approach to genotoxicity assessment procedures by employing a genetically engineered cell line to report on a number of key indicators of genotoxicity effects and mechanisms. Imaging and analysis of cells exposed to test agents allows automated analysis using high content cellular screening to identify cytostatic and/or cytotoxic activity, to quantify micronuclei formation, to discriminate aneugenic and clastogenic micronuclei and to detect DNA mutation and repair.

### BACKGROUND OF THE INVENTION

The present invention relates to the field of testing new chemical entities for genotoxicity and specifically to methods for determining genotoxicity by high-throughput automated cellular imaging and analysis.

### FIELD OF THE INVENTION

Genotoxicity is generally defined as any damage to the integrity of a cell's DNA, caused either directly through chemical interaction with DNA, or indirectly through interference with the cell's structural or enzymatic machinery, that has the potential to lead to an inheritable defect in the genetic information carried by that cell.

Regulatory guidelines require that all registered drugs undergo assessment for genotoxicity in a panel of assays. These assays include measurement of gene mutation in bacteria and determination of micronuclei formation in-vitro and in-vivo. The assays are typically extremely time consuming and expensive to perform (˜$50,000/compound), and thus tend to be restricted to the end of the drug discovery process. Estimates of the cost of failure or inability to accurately predict toxicity early in drug development have been put as high as $8 billion/year, around 30% of all drug failure costs (Cambridge Healthtech Advances Life Sciences Report Dec 2004: Toxicogenomics and Predictive Toxicology: Market and Business Outlook). In addition to testing of new drugs, there is increasing regulatory activity to extend genotoxicity testing to other chemicals and materials, such as food dyes and cosmetics that are intended for human consumption or use, or which may be indirectly or accidentally consumed or ingested (OECD guideline for the testing of chemicals, draft proposal for a new guideline 487, June 2004).

Among the reasons for the high costs of genotoxicity testing is the number of separate testing procedures required and the high level of skilled manual effort involved in performing these procedures. For example, one of the most commonly performed genotoxicity test, the in vitro micronucleus assay, may require 1-2 weeks of operator time for manual scoring. As shown in FIG. 1, the in vitro micronucleus assay detects genotoxicity by incubating cells in tissue culture for 24-72 hours in the presence of test compounds. During mitosis, the process by which cells divide and proliferate, the division between the separate cytoplasmic [1] and nuclear [2] compartments of the cell break down to allow the cellular DNA carried on chromosomes to be segregated between the two daughter cells. In the mitotic cell [3] chromosomes [4] are mechanically separated to either end of the cell by the action of microtubules [6] attached to spindle poles [5] at either end of the cell and to centromeres [7] on the chromosomes. Micronuclei formation may occur during cell division of cells exposed to genotoxic compounds through two mechanisms. Clastogenic agents cause DNA strand breakage [8] leading to detachment of part of a chromosome from the microtubule machinery. In contrast, aneugenic agents interfere with chromosome segregation by inhibiting microtubule function [9]. Either of these mechanisms can lead to incorrect segregation of DNA between the two daughter cells formed after mitosis, such that once the nuclear and cytoplasmic compartments of the cell are formed one daughter cell [10] has the correct complement of DNA, while the other daughter cell [11] has a DNA deficient nucleus [12] with the remainder of the DNA contained in a micronucleus [13] in the cell cytoplasm. To assess genotoxicity cellular DNA, including any DNA in micronuclei, is stained with a fluorescent dye, cells are imaged and the frequency of micronuclei formation quantified.

In some cases it is desirable to distinguish between clastogenic and aneugenic mechanisms of micronuclei formation. For example, in drug development, segregating compounds into clastogenic and aneugenic classes offers a means for prioritising candidates for further development. However, such mechanistic distinctions require additional complex and time consuming procedures. Fluorescence in-situ hybridisation (FISH) methods (Nersesyan, A., et al., Anticancer Drugs, (2006), 17(3), 289-95) with all-chromosome centromeric nucleic acid hybridisation probes allow discrimination between clastogenic micronuclei formed from chromosomal fragments (centromere negative FISH) and aneugenic micronuclei containing whole chromosomes (centromere positive FISH). Alternatively, antibody staining of micronuclei for centromere or kinetochore proteins may be used to distinguish aneugenic and clastogenic micronuclei (Parry, E. M. et al., Mutagenesis (2002) 17(6), 509-21).

While the Salmonella mutagenicity assay, commonly known as the Ames test (Seifried, H. E., et al., Chem.Res.Toxicol., (2006), 19(5), 627-44), remains the current regulatory approved base method of assessing compound mutagenicity, developments using engineered yeast (Billinton, N. et al., Biosens.Bioelectron, (1998), 13(7-8), 831-8) have been proposed as a complementary or alternative approach. GreenScreen (Gentronix) is a genotoxicity and cytotoxicity assay based on a yeast strain engineered to express green fluorescent protein (GFP) under the control of a DNA repair gene promoter (RAD54). Cells with DNA damage as a result of exposure to genotoxic compounds show increased GFP fluorescence relative to control cells.

Detection of DNA damage and mutation in mammalian cells has been reported previously (Todd, et al., Fund. Appl. Toxicol., (1995), 28, 118-128) using chloramphenicol acetyl transferase (CAT) as a reporter gene under the control of the p53 responsive promoter for GADD45a (Growth Arrest and DNA Damage gene). However, measurement of CAT activity in cells is a destructive process, precluding the combination of GADD45a-CAT induction with other cellular measurements such as micronuclei formation.

A recently published development of this approach has extended the use of a DNA damage sensor to mammalian cells where expression of GFP is under the control of the GADD45a promoter (Hastwell, P. W., et al., Mutat.Res., (2006), 607(2), 160-75). However, the use of GFP as a reporter potentially limits the sensitivity of the assays as the reporter gene signal is not generated by enzymatic amplification. Furthermore, the use of GFP as the reporter for DNA damage or mutation precludes combining this assay with discrimination of aneugenic and clastogenic genotoxicity by marking chromosome centromeres with GFP.

Consequently none of the above methods described above are suitable for combination into an integrated single mammalian cell based assay capable of yielding data on micronuclei formation, distinguishing clastogenic and aneugenic activity, and DNA mutation.

Recent developments in high-content screening (HCS) platforms, such as INCELL™ Analyzer 1000 (GE Healthcare), have allowed automation of image acquisition and data analysis for cell based assays and therefore have significant potential to automate genotoxicity testing. Automation of these assays and the subsequent reduction in time and cost, through the removal of manual scoring, has the potential to allow larger numbers of compounds to be tested for genotoxic effects. Within the pharmaceutical industry, this has the potential to allow testing of new chemical entities at an earlier stage in drug development than possible with traditional approaches, with associated savings by highlighting potential genotoxic compounds prior to expenditure of time and resources in medicinal chemistry programs. In other areas, such as in the development of new dyes for foodstuffs, cosmetics and other domestic products the availability of lower cost automated genotoxicity assessment will serve to offset rising costs associated with increased testing demanded by regulatory authorities.

In addition to the potential for HCS technology to automate single genotoxicity assays, the multi-parameter nature of the analytical process lends itself to a new approach to genotoxicity testing wherein the technology is used to provide data from a single assay procedure where previously the same data would have required a battery of separate tests. For example, automated analysis of micronuclei formation using HCS provides additional data on cytostatic or cytotoxic activity by determining the cell proliferation index of cultures exposed to test compounds.

Consequently, integrating separate genotoxicity assays by designing individual readouts to be compatible with multiplexing in a single cell and using HCS technology to image and analyse such readouts provides the ability to generate more highly informative genotoxicity assessments in a more cost effective manner.

It has been demonstrated previously that multiplexing GFP and reporter gene based assays may be achieved in the same cell without one assay interfering with the other (GE Healthcare Application note 28-9040-13 AA; Multiplex assays using AD-A-GENE™ Vectors: EGFP glucocorticoid receptor and glucocorticoid response element—nitroreductase). This work demonstrates that monitoring the agonist induced translocation of a glucorticoid receptor fused to GFP from the cell cytoplasm to the nucleus can be achieved in the same cell as measuring the subsequent activation of a nitroreductase reporter gene coupled to a glucocorticoid response element. Translocation of the glucocorticoid receptor from the nucleus produces an increase in reporter gene activity measured by conversion of a non-fluorescent substrate to a red fluorescent product. The demonstration of non-interfering nature of these two cellular sensor systems permits the use of the same independent multiplexable sensors in the method of the present invention to monitor centromere protein distribution via a GFP fusion protein and DNA damage via a nitroreductase reporter gene. What has not been described previously is a multiplexed GFP and reporter gene based assay in the same cell where the two outputs monitor two or more independent processes rather than two sequential and dependent processes.

### SUMMARY OF THE INVENTION

Disclosed are compositions and methods which provide an integrated approach to genotoxicity assessment procedures by employing a genetically engineered cell line to report on a number of key indicators of genotoxicity effects and mechanisms. Imaging and analysis of cells exposed to test agents allows automated analysis using high content cellular screening to identify cytostatic and/or cytotoxic activity, to quantify micronuclei formation, to discriminate aneugenic and clastogenic micronuclei and to detect DNA mutation and repair.

According to a first aspect, there is provided a cell comprising:
a) a first reporter gene construct comprising a nucleic acid molecule encoding a fluorescent protein fused to a centromere protein said nucleic acid sequence operably linked to and under the control of a promoter that is constitutively active; and
b) a second reporter gene construct comprising a nucleic acid molecule comprising one or more expression control sequences operably linked to a sequence encoding a nitroreductase (NTR) enzyme.

Preferably, the expression control sequences comprise a DNA damage induced promoter; more preferably the promoter is GADD45a.

Thus, a cell line is disclosed comprising at least one cell that is engineered to a) constitutively express a fluorescent protein in fusion with a centromere protein; and b) inducibly express a nitroreductase (NTR) enzyme reporter gene under the control of expression control sequences, suitably a DNA damage induced promoter.

Suitable promoters that allow constituitive expression of a fluorescent protein may be selected from CMV, Ubiquitin C, Ferritin, Transferrin, SV40, EF1α, and others well known to those skilled in the art to be suitable for constitutive expression of proteins in mammalian cells.

Preferably, the centromere protein is centromere protein A (CENP-A).

In a second aspect, there is provided a process for producing a cell line including at least one cell comprising first and second reporter gene constructs capable of being expressed. The process comprises:
a) transfecting one or more cells with a first and a second recombinant expression vector, wherein:
   i) said first recombinant expression vector comprises a first reporter gene construct comprising a nucleic acid molecule encoding a fluorescent protein fused to a centromere protein said nucleic acid sequence operably linked to and under the control of a promoter that is constitutively active; and
   ii) said second recombinant expression vector comprises a second reporter gene construct comprising a nucleic acid molecule comprising one or more expression control sequences operably linked to a sequence encoding a nitroreductase (NTR) enzyme; and
b) selecting transfected cells which express at least one and optionally both first and second reporter genes.

Preferably, the at least one cell is transfected with the first recombinant expression vector followed by the second recombinant expression vector. By using this method, cells are transfected with a suitable expression vector encoding the fluorescent centromere fusion and the resulting cells cloned by limiting dilution and screened to select cell clones in which expression of fluorescent protein is at a level suitable for detection by automated imaging. Selected cell clones can be further screened by exposure to known clastogens (e.g. Mitomycin C) and known aneugens (e.g. Vincristine), and one or more clones selected that exhibit fluorescent protein localisation in micronuclei specific to treatment with aneugens. Such selected clone(s) of fluorescent fusion protein expressing cells are then transfected with a suitable expression vector comprising a nucleic acid molecule comprising one or more expression control sequences operably linked to a sequence encoding a nitroreductase. The resultant cells can then be further screened to isolate clones in which nitroreductase expression is induced by treatment(s), known to induce DNA damage and/or repair, e.g. methyl methane sulphonate or ionising radiation.

Suitably, the engineered cell line according to the first aspect is a eukaryotic cell line, preferably a mammalian cell line which may be normal or transformed, for example a human, rodent, or simian derived cell. Examples of suitable recombinant host cells include, but are not restricted to, HeLa cells, Vero cells, Chinese Hamster ovary (CHO), U2OS, COS, BHK, HepG2, NIH 3T3 MDCK, RIN, HEK293 and other mammalian cell lines that are grown in vitro. Such cell lines are available from the American Tissue Culture Collection (ATCC), Bethesda, Md., U.S.A.

As disclosed herein, the term “operably linked” indicates that the elements comprising the reporter gene constructs are arranged such that they function in concert for their intended purposes. For example, the first reporter gene construct is arranged such that transcription initiates in a promoter and proceeds through the DNA sequence coding for the fluorescent protein.

In a third aspect, there is provided an assay method for determining the effect of a test agent on a phenotypic property of a cell. The method comprises:
a) providing cells comprising:
   i) a first reporter gene construct comprising a nucleic acid molecule encoding a fluorescent protein capable of emitting fluorescent light of a first wavelength (λ₁) and being fused to a centromere protein said nucleic acid sequence operably linked to and under the control of a promoter that is constitutively active; and
   ii) a second reporter gene construct comprising a nucleic acid molecule comprising one or more expression control sequences operably linked to a sequence encoding a nitroreductase (NTR) enzyme;
   and wherein said cells are marked with a DNA staining dye that emits fluorescent light of a second wavelength (λ₂) when bound to DNA;
b) contacting a population of said cells with:
   iii) a test agent or a control; and
   iv) a substrate for nitroreductase wherein said substrate is capable of emitting fluorescent light of a third wavelength (λ₃);
      under conditions permitting expression of said first and second reporter gene constructs; and
c) measuring the fluorescence intensity at different wavelengths of emitted light in said cell population;
   wherein a difference in fluorescence intensity in cells treated with said test agent compared with cells treated with said control is indicative of the effect of said test agent on the phenotypic property of said cell.

Suitably, the first wavelength (λ₁), the second wavelength (λ₂) and the third wavelength (λ₃) of emitted light are each different and distinguishable one from the other.

Preferably, the phenotypic property may be selected to be cell number, nuclear morphology, micronuclear frequency, centromere status of micronuclei and reporter gene expression.

Preferably, cells for use in the disclosed method are eukaryotic cells, more preferably mammalian cells.

Suitably, according to the invention, the fluorescent protein may be selected from group consisting of wild type Green Fluorescent Protein (GFP) from Aequorea Victoria and derivatives of GFP, such as functional GFP analogues. Preferred fluorescent proteins for use in the disclosed method include EGFP (Cormack, B. P. et al., Gene, (1996), 173, 33-38); EYFP and ECFP (U.S. Pat. No. 6,066,476, Tsien, R. et al.); F64L-GFP (U.S. Pat. No. 6,172,188, Thastrup, O. et al.); BFP, (U.S. Pat. No. 6,077,707, Tsien, R. et al.). Other fluorescent proteins include NFP (Clontech) and Renilla GFP (Stratagene).

Suitably, the expression control sequences operably linked to the nitroreductase gene comprise a DNA damage induced promoter, which may be selected from GADD45a, GADD45p, p53, hOgg1, JUN, FOS, GADD153, MGMT. Preferably, the promoter is GADD45a.

Suitably, the nitroreductase substrate employed in the assay method is a dye comprising at least one NO₂, preferably a cyanine dye or a squaraine dye. In particularly preferred embodiments, the cyanine dye or squaraine dye is cell permeable.

In one embodiment of the disclosed method, cells are cultured in the presence of one or more test agents, stained with a DNA dye (e.g. Hoechst or DAPI) and a NTR substrate, and imaged using automated microscopy to acquire images of DNA (blue fluorescence), GFP-centromere fusion protein (green fluorescence) and NTR (red fluorescence). The resulting images are processed using automated image analysis algorithms to segment and identify nuclei, segment and count micronuclei, detect the presence of centromeres in micronuclei, measure reporter gene expression and make a number of additional morphological measures to assess cell viability and proliferation.

In further embodiments, the fluorescent colours of the components of the engineered cell line are varied according to different combinations of imaging in blue, green and red wavelengths. Such embodiments include, but are not restricted to, the use of red fluorescent protein to mark centromeres and a green fluorescent substrate for the NTR reporter gene assay. It will be appreciated by those skilled in the art that, by varying the colours of the DNA stain, the fluorescent protein and the NTR substrate, while keeping each read out spectrally distinct, that a variety of colour combinations can be achieved for the multiparameter genotoxicity cellular assay as disclosed herein.

### BRIEF DESCRIPTION OF THE DRAWINGS

FIG. 1 shows a schematic of the mechanism of micronuclei formation induced by clastogenic and aneugenic genotoxic agents.

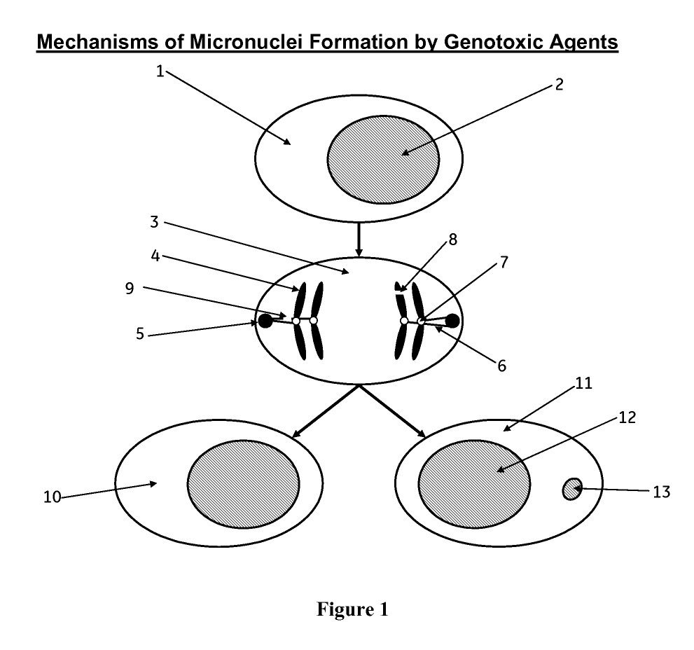

FIG. 2 shows multiparameter genotoxicity cell assay phenotypes detected by the assay method according to the invention.

FIG. 3 shows an analysis algorithm for the multiparameter genotoxicity cell assay according to the invention.

### DETAILED DESCRIPTION OF THE INVENTION

To enable discrimination of micronuclei formed via clastogenic and aneugenic mechanisms, the cell line of the invention comprises a fusion protein between a fluorescent protein (FP) and a protein chosen to be located in the centromere region of all cell chromosomes. The histone H3-variant, centromere protein A (CENP-A) comprises the primary link between centromeric DNA and the protein components of the kinetochore. Stable expression of GFP-CENP-A (Irvine, D. V. et al., Chromosome Res., (2004), 12(8), 805-15) has shown that this fusion protein is incorporated into chromosomes and localises to the inner kinetochore plate of active centromeres. Expression of this fusion protein in a stable cell line used in a standard in-vitro micronucleus protocol permits clastogenic and aneugenic mechanisms to be distinguished by HCS analysis with no requirement for FISH or antibody staining. Dual colour image analysis allows micronuclei to be identified by fluorescent DNA staining and imaging DNA and the FP fusion protein, with subsequent classification of micronuclei as aneugenic (FP positive micronuclei) or clastogenic (FP negative micronuclei).

Suitable fluorescent proteins for use in the present invention include blue fluorescent proteins (U.S. Pat. No. 6,077,707), green fluorescent proteins and red fluorescent proteins (Tsien, R. et al., Nat.Methods, (2005), 2(12), 905-9). Preferred fluorescent proteins include wild type GFP from Aequorea Victoria and derivatives of GFP such as functional GFP analogues in which the amino acid sequence of wild type GFP has been altered by amino acid deletion, addition, or substitution. Such fluorescent proteins include EGFP (Cormack, B. P. et al., Gene, (1996), 173, 33-38); EYFP and ECFP (U.S. Pat. No. 6,066,476, Tsien, R. et al.); F64L-GFP (U.S. Pat. No. 6,172,188, Thastrup, O. et al.); BFP, (U.S. Pat. No. 6,077,707, Tsien, R. et al.). Other fluorescent proteins include NFP (Clontech) and Renilla GFP (Stratagene).

The construction and use of expression vectors to achieve stable expression of a fluorescent fusion protein in mammalian cells are well known to those skilled in the art. Virtually any mammalian cell expression vector may be used to generate the expression of stable marked chromosome centromeres as described herein. Examples of suitable vector backbones which include mammalian drug resistance genes for selection of expressing cells include, but are not limited to pCI-neo (Promega), pcDNA (Invitrogen), pTriEx1 (Novagen) and PCORON (GE Healthcare). The DNA construct encoding the FP-centromere protein fusion can be prepared by standard molecular biology techniques of polymerase chain reaction (PCR) amplification, restriction enzyme digestion, ligation, transformation and plasmid purification which are familiar to those skilled in the art and are fully described in standard textbooks (for example in Sambrook et al. (1989), Molecular Cloning—A Laboratory Manual, Cold Spring Harbor Press). DNA coding sequences for centromere proteins are readily obtained by PCR from a cDNA library (Irvine, D. V., et al., Chromosome Res., (2004), 12(8), 805-15). To provide stable expression of the fluorescent fusion protein, a constitutively active promoter such as the human cytomegalovirus (CMV) immediate early promoter (Thomsen, et al., Proc. Natl. Acad. Sci. USA, (1984), 81(3), 659-63), or the Ubiquitin C promoter (Schorpp et al., Nucleic Acids Res., (1996), 24(9), 1787-8) can be used to drive expression of the fusion protein. The vector containing the fluorescent fusion protein and associated regulatory sequences can be introduced into the host cell by transfection using well known techniques, for example by use of DEAE-Dextran or Calcium Phosphate (Molecular Cloning, A Laboratory Manual 2nd Edition, Cold Spring Harbour Laboratory Press (1989) pp 16.30-16.46). Other suitable techniques using lipid based transfection such as Lipofectamine (Invitrogen) will be well known to those skilled in the art. Alternatively, viral vectors, including but not limited to Lentivirus (Kafri et al., Nat. Genet., (1997), 17(3), 314-7; Lois et al., Science, (2002), 295(5556), 868-72) or Retrovirus (Lybarger et al., Cytometry, (1996), 25(3), 211-20; Levy et al., Nat. Biotechnol., (1996), 14(5), 610-4) can be used to establish stable integration of fusion protein coding sequences into mammalian cells.

To enable the detection of genotoxic DNA damage and/or mutation not giving rise to the formation of micronuclei, the cell according to the present invention further comprises a DNA damage reporter gene compatible with detection in intact cells. The nitroreductase (NTR) enzyme gene reporter system (EP 1252520 B1) comprises a method for increasing the fluorescence of a cyanine dye molecule comprising at least one NO₂ group, characterised by the reduction of the at least one NO₂ group to NHOH or NH₂ by the action of a nitroreductase. A quenched cyanine dye (Cy-Q) or squaraine dye molecule comprising at least one NO₂ group may be used as a substrate for detecting nitroreductase enzyme activity and allows for the use of a nitroreductase enzyme in an enzyme-reporter system for the detection of gene expression. Depending on the structure of the cyanine or squaraine dye substrate, the fluorescence emission from the product of the NTR reaction can occur across a wide range of wavelengths, typically 500-900 nm. Moreover, the fluorescence emission characteristics of the reduced dye-product of the NTR reaction can be altered to suit the application by making changes to the internal structure of the dye molecule, without changing the extremities of the molecule, e.g. the NO₂ groups that are involved in the reaction with nitroreductase. This range of emission wavelengths is particularly advantageous for multiplexing detection of cellular NTR gene reporter activity in combination with other fluorescence readouts in the same cell.

Preferred nitro group-containing dyes for use as NTR substrates in the disclosed assay method are dyes having the general formula:

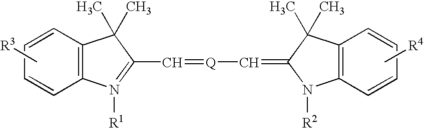

wherein:
Q is a fragment selected from the group consisting of:

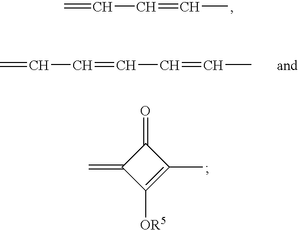

R¹ and R² are independently selected from C₁-C₄ alkyl, -(CH₂)ₙ—P,—{(CH₂)₂—O}ₚ—R⁶ and the group W; where P is selected from COOR⁷, SO₃⁻ and OH, W is mono- or di-substituted nitrobenzyl, R⁶ is methyl or ethyl, R⁷ is selected from H, C₁-C₄ alkyl and CH₂OC(O)R⁸, where R⁸ is methyl, or t-butyl, n is an integer from 1 to 10, and p is an integer from 1 to 3;
groups R³ and R⁴ are independently selected from hydrogen, NO₂, halogen, SO₃⁻, C₁-C₄ alkoxy and -(CH₂)ₘ—COOR⁷; where R⁷ is hereinbefore defined and m is 0 or an integer from 1 to 5;
R⁵ is hydrogen or C₁-C₄ alkyl;
provided that at least one of groups R¹, R², R³ and R⁴ comprises at least one NO₂ group.

As disclosed herein, the sulphonate group (SO₃⁻) is also intended to represent the sulphonic acid group (SO₃H), since sulphonate is the ionised form of the parent acid.

Preferably, compounds according to the above formula are those wherein one of groups R¹ and R² is selected from group W where W is selected from:

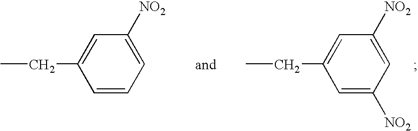

and remaining R¹ or R² is selected from methyl, ethyl, the group -{(CH₂)₂—O}ₚ—R⁶ and the group -(CH₂)ₙ—COOR⁷, where R⁶ is methyl or ethyl, and R⁷ is H or C₁-C₄ alkyl. Particularly preferred compounds are those wherein W is the group:

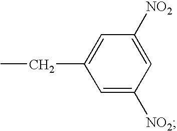

and remaining R¹ or R² is hereinbefore defined. Such compounds are described in European Patent Application No. 1086179 A1 (Hamilton, A. et al.) or in International Patent Application No.WO 2005/118839 (West, R. M. et al.).

Particular examples of NTR substrates useful in the assay method are CY5Q™ and 2-(1-(5-carboxypentyl)-3,3-dimethyl-2-indolinylidenemethyl)-4-(1-(3,5-dinitrobenzyl)-3,3-dimethyl-2-indolinylidenemethyl)cyclobutenediylium-1,3-diolate (CytoCy5S) (GE Healthcare).

Methods for using enzyme genes as reporter genes in mammalian cells are well known (for review see Naylor L. H., Biochemical Pharmacology, (1999), 58, 749-757). The reporter gene is chosen to allow the product of the gene to be measurable in the presence of other cellular proteins and is introduced into the cell under the control of a chosen regulatory sequence which is responsive to changes in gene expression in the host cell. Typical regulatory sequences include those responsive to hormones, second messengers and other cellular control and signalling factors. For example, agonist binding to seven transmembrane receptors is known to modulate promoter elements including the cAMP responsive element, NFAT, SRE and AP1; MAP kinase activation leads to modulation of SRE leading to Fos and Jun transcription; DNA damage leads to activation of transcription of DNA repair enzymes and the tumour suppressor gene p53. By selection of an appropriate regulatory sequence the reporter gene can be used to assay the effect of added agents on cellular processes involving the chosen regulatory sequence under study.

For use as a reporter gene, the nitroreductase gene can be isolated by well known methods, for example by amplification from a cDNA library by use of the polymerase chain reaction (PCR) (Molecular Cloning, A Laboratory Manual 2nd Edition, Cold Spring Harbour Laboratory Press (1989) pp 14.5-14.20). Once isolated, the nitroreductase gene can be inserted into a vector suitable for use with mammalian promoters (Molecular Cloning, A Laboratory Manual 2nd Edition, Cold Spring Harbour Laboratory Press (1989) pp 16.56-16.57) in conjunction with and under the control of the gene regulatory sequence under study. The vector containing the nitroreductase reporter and associated regulatory sequences can then be introduced into the host cell by transfection using well known techniques, as previously described herein for introduction of sequences encoding fluorescent fusion proteins.

To determine NTR reporter gene activity cells are incubated in the presence of a membrane permeable NTR substrate such as CytoCy5S (GE Healthcare) to allow conversion of the quenched substrate to its fluorescent form. Imaging cells incubated with this substrate using an excitation wavelength of 628 nm, and an emission wavelength of 638 nm allows measurement of NTR activity in the presence of blue and green fluors, for example Hoechst and GFP respectively, without spectral interference.

Engineering the cell line of the present invention with a GADD45a-NTR reporter gene additionally allows the detection of genotoxic or mutagenic activity not resulting in micronuclei formation. DNA damage resulting from exposure to a test agent results in activation of the GADD45a-NTR via p53, and the resulting increase in NTR expression is monitored by detecting an increase in fluorescence emitted from the substrate, CytoCy5S.

When exposed to test agents, the cell line described herein can exhibit a variety of phenotypes that are detectable by automated microscopy using suitable HCS instrumentation. Suitably, the test agent can include any chemical entity which is added to cells in tissue culture, such as a drug, a food dye, a hormone, a toxin, an alkylating agent, an oxidising agent, a carcinogen. Alternatively, the test agent can be a non-chemical entity, i.e. a physical agent or physical treatment which may be applied to cells in culture, such as electromagnetic radiation (e.g. UV, X-ray, microwave), β⁻ radiation, heat.

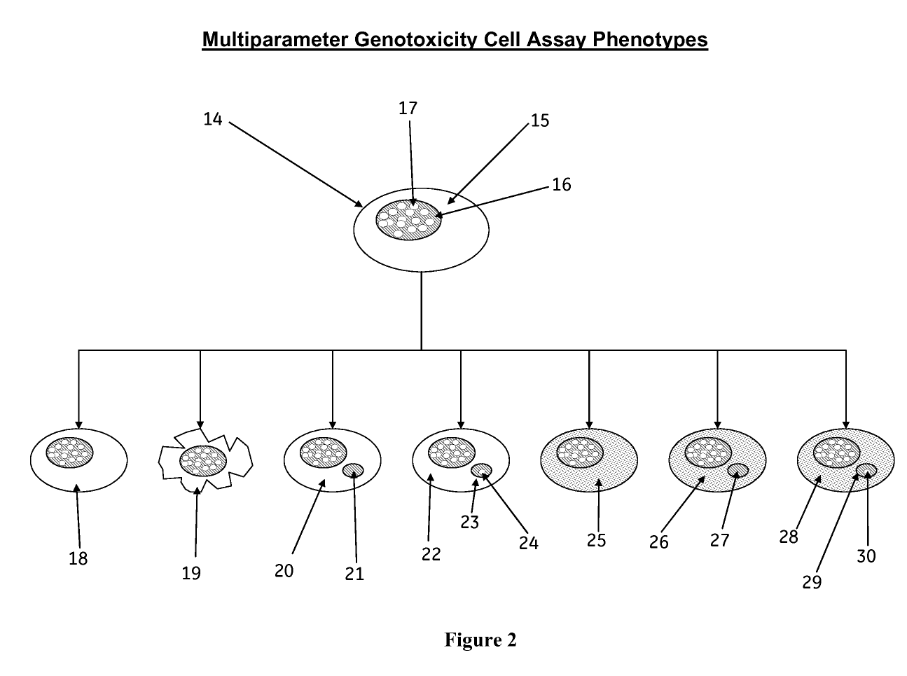

With reference to FIG. 2, the cell [14] comprises cytoplasm [15] and nucleus [16] wherein the nucleus contains chromosomes tagged with a fluorescent centromere fusion protein detectable by fluorescent imaging [17]. Exposure of cells to a test agent (or control) having no toxic or genotoxic activity does not alter the phenotypic appearance of cells [18]. However, test compounds that inhibit (or stimulate) cell proliferation are detectable by a change in cell number relative to cells grown in the absence of the test compound or a control. Compounds with cytotoxic activity produce detectable changes in cellular morphology [19] associated with breakdown of nuclear and/or cytoplasmic structure. Cells exposed to clastogenic compounds [20] can form micronuclei [21] detectable by imaging DNA stained with a suitable fluorescent dye. Cells exposed to compounds with aneugenic activity [22] can form micronuclei [23] where the micronuclei contain a centromere [24] detectable by imaging of the incorporated fluorescent centromere fusion protein. Cells exposed to a compound giving rise to DNA mutation and/or repair [25] where the compound does not exhibit clastogenic or aneugenic activity show increased reporter gene activity detectable by imaging of a fluorescent substrate. Cells exposed to a compound having both clastogenic and mutagenic properties show both up-regulation of the reporter gene [26] and micronuclei formation [27]. Cells exposed to a compound having both aneugenic and mutagenic properties show both up-regulation of the reporter gene [28] and micronuclei formation [29] with micronuclei containing fluorescent centromeres [30].

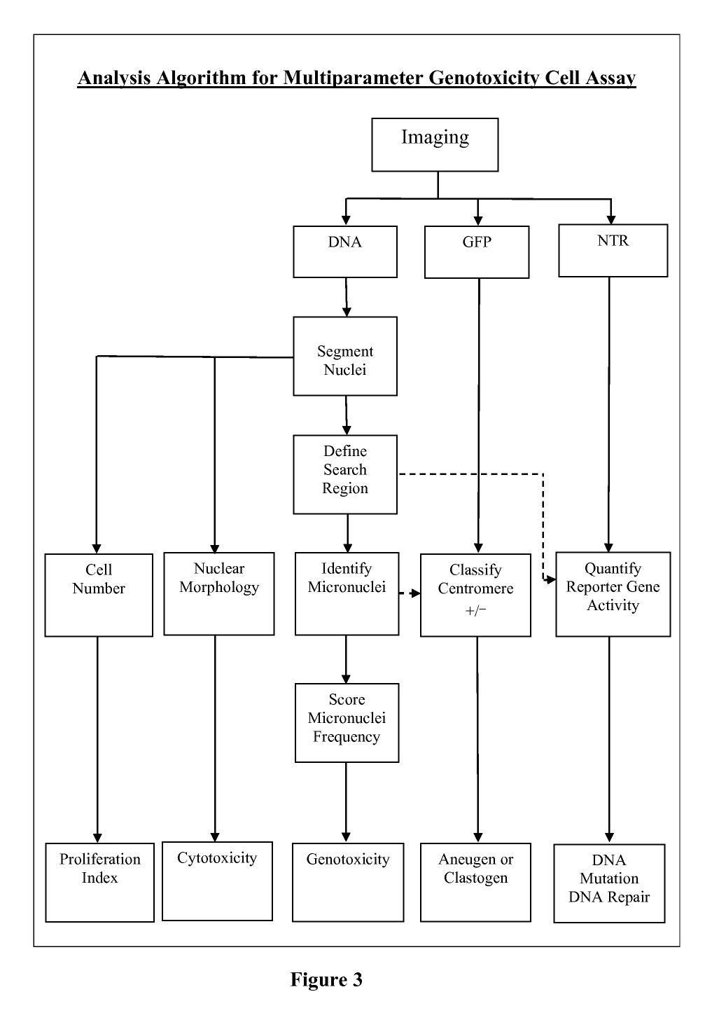

Classification of cell phenotypes reflecting the activity of test compounds is readily achieved using suitable image analysis software such as Multi-Target Analysis (GE Healthcare). With reference to FIG. 3, showing the image analysis process for one illustrative embodiment, fluorescence imaging of cells is performed in three colours, corresponding to the fluorescence from DNA (Blue), a centromere-GFP fusion protein (Green) and NTR substrate conversion (Red).

The DNA (Blue) image is used to segment the image to identify all nuclei (and therefore cells) in the image. This process allows cell number to be determined and the abstraction of a number of nuclear and cellular morphology parameters. Nuclear segmentation is then used to define a region around the nucleus in which to search for the presence of micronuclei. Micronuclei are identified by secondary segmentation of the DNA (Blue) image within the search area to identify extra-nuclear DNA, i.e. micronuclei. Regions of the DNA image identified as being micronuclei are then examined in the GFP (Green) image (indicated by the dotted line), to determine whether micronuclei have GFP fluorescence indicative of the presence of centromeres. In addition, the search region is applied to the NTR (Red) image, (indicated by the dotted line), to measure the intensity of fluorescent substrate conversion in each cell as a measure or reporter gene activity.

Once these data abstraction steps have occurred cells can be classified into different phenotypic categories, including but not limited to, normal morphology, aberrant morphology, micronuclei positive, micronuclei negative, centromere positive micronuclei, centromere negative micronuclei, reporter gene positive, and reporter gene negative to provide a multiparameter genotoxicity phenotypic signature for each test compound.

In a further aspect, a diagnostic kit is provided said kit comprising:
a) at least one cell comprising:
   i) a first reporter gene construct comprising a nucleic acid molecule encoding a fluorescent protein fused to a centromere protein said nucleic acid sequence operably linked to and under the control of a promoter that is constitutively active; and
   ii) a second reporter gene construct comprising a nucleic acid molecule comprising one or more expression control sequences operably linked to a sequence encoding a nitroreductase (NTR) enzyme; and
b) a nitroreductase (NTR) enzyme substrate.

Suitably, the nitroreductase substrate is a dye comprising at least one NO₂, preferably a cyanine dye or a squaraine dye. In particularly preferred embodiments the cyanine or squaraine dye is cell permeable.

### EXAMPLES

The following examples serve to illustrate the processes according to embodiments of the present invention and are not intended to be limiting.

### Example 1

### Dual Reporter Cell Line for Genotoxicity Assays.

Human U2OS osteosarcoma cells (ATTC code HTB-96) are maintained under standard (37° C./5%CO₂) tissue culture conditions in Dulbecco's Modified Eagles medium (Sigma-Aldrich) supplemented with 10% calf serum (Sigma-Aldrich). PCR primers (Irvine, D. V. et al., Chromosome Res., (2004), 12(8), 805-15) are used to amplify the coding sequence for human CENP-A centromere protein gene from a cDNA plasmid (Origene #SC121879) using ILLUSTRA™ RTG PCR beads (GE Healthcare #27-9557-01) according to the manufacturer's instructions. The PCR product is purified using an ILLUSTRA™ Microspin S-400HR column (GE Healthcare #27-5140-01 according to the manufacturer's instructions. The purified DNA is cut with Xhol and EcoRI restriction enzymes (New England Biolabs) and cloned into the multiple cloning site of a pCORON-GFP-C1 mammalian expression vector (GE Healthcare) under the control of a CMV promoter using standard techniques (Molecular Cloning, A Laboratory Manual 2nd Edition, Cold Spring Harbour Laboratory Press (1989) pp 16.56-16.57). U2OS cells are passaged from stock culture into 6 well dishes at 3×10⁵ cells/well and grown overnight to 80% confluency. Cells are transfected with CENPA-GFP plasmid using a transfection mix comprising 1 μg plasmid and 3 μl Fugene 6 lipid transfection reagent (Roche #11-815-091-001) for each well. Cells are placed under chemical selection using G418 (Sigma Aldrich) at 500 μg/ml and monitored for GFP expression by fluorescence microscopy and flow cytometry. GFP exhibiting stable expression of CENPA-GFP are maintained under standard tissue culture conditions under G418 selection and clones showing punctuate fluorescence indicative of correct localisation of the CENPA-GFP fusion protein to centromeres are cloned by limiting dilution in 96 well plates for further processing. The DNA sequence for the GADD45a p53 regulated DNA damage responsive promoter is amplified from human genomic DNA using PCR primers as previously described (Hastwell, P. W., et al., Mutat.Res., (2006), 607(2), 160-75 and WO2005/113802), and ILLUSTRA™ RTG PCR beads (GE Healthcare #27-9557-01) according to the manufacturer's instructions. The PCR product is purified using an ILLUSTRA™ Microspin S-400HR column (GE Healthcare #27-5140-01 according to the manufacturer's instructions. The purified amplified DNA is cloned into a nitroreductase (NTR) expression vector (GE Healthcare #25-9902-00) supporting hygromycin (Sigma-Aldrich) selection of stable reporter gene expression such that the NTR is under the control of the GADD45a promoter. U2OS cells previously selected to have stable CENPA-GFP expression are transfected with the GADD45a-NTR vector as described above for CENPA-GFP transfection. Cells are subsequently maintained under standard tissue culture conditions with G418 (500 μg/ml) and hygromycin (200 μg/ml) selection to select for cells with stable incorporation of both CENPA-GFP and GADD45a-NTR sequences. Cells are cloned by limiting dilution in 96-well plates and clones expanded by growth under dual chemical selection and frozen in culture media supplemented with 10% DMSO until use.

For assay of test agents for genotoxicity CENPA-GFP/GADD45a-NTR cells are grown in 96-well imaging plates (Packard) in 100 μl Dulbecco's Modified Eagles medium supplemented with 10% calf serum and exposed to test agents for 48 hours. To detect NTR activity cells are incubated for 4 hours at 37° C. with 1 μM CytoCy5-S NTR substrate (GE Healthcare #PA76140) followed by washing with 200 μl phosphate buffered saline pH 7.4 and fixation with 4% formalin solution. Nuclear DNA is stained with 2.5 μM Hoechst 33342 (Invitrogen) for 15 minutes at room temperature. Cells are imaged using INCELL™ Analyzer 1000 (GE Healthcare) using three colour image acquisition with a 360/40-nm excitation filter (Hoechst), 475/20-nm excitation filter (GFP) and 535/20-nm emission filter (GFP and Hoechst), and 620/60-nm excitation and 700/75-nm emission filters (NTR CytoCy5-S product). Images are analyzed using INCELL™ Investigator MTA analysis software (GE Healthcare) using decision tree analysis to determine the effect of the test agents on cell proliferation, cell morphology, micronuclei induction, CENPA-GFP localisation and NTR reporter gene expression (FIG. 3).

By combining two independently operating reporter systems in the same cell, the method of the present invention confers a number of advantages over prior art methods. Firstly, by combining multiple means of assessing DNA damage in a single assay the method of the present invention provides benefits in increased throughput and reduced usage of test agent relative to performing separate assays for DNA damage. Secondly, since the assay read-outs are operating independently in the same cells the method of the present invention provides means to correlate DNA damage indicators at the individual cell level which is not possible when assays are performed separately. For example using the method of the present invention allows the user to determine in any given cell whether a test agent is active solely through an aneugenic mechanism (as reported by GFP localisation to micronuclei), causing generalised DNA damage (as reported by an increase in NTR reporter gene activity) or both, and to determine whether any activity of a test agent in causing DNA damage varies in a cell population due to underlying heterogeneity in the population caused by inherent biological variation, for example cell cycle position, where a sub-population of cells may be more or less susceptible to damage arising from the test agent.

While preferred illustrative embodiments of the present invention are described, one skilled in the art will appreciate that the invention can be practiced by other than the described embodiments. The following example is presented to illustrate how the invention may be practised and not by way of limitation. The present invention is limited only by the claims that follow.

What is claimed is:

1. A cell comprising:
a) a first reporter gene construct comprising a nucleic acid molecule encoding a fluorescent protein fused to a centromere protein said nucleic acid sequence operably linked to and under the control of a promoter that is constitutively active; and
b) a second reporter gene construct comprising a nucleic acid molecule comprising one or more expression control sequences operably linked to a sequence encoding a nitroreductase (NTR) enzyme.

2. The cell of claim 1, wherein said centromere protein is centromere protein A (CENP-A).

3. The cell of claim 1, wherein said fluorescent protein is selected from Green Fluorescent Protein (GFP) or a functional GFP analogue.

4. The cell of claim 3, wherein said fluorescent protein is selected from wild type GFP, EGFP, EYFP, ECFP, F64L-GFP, BFP, NFP and Renilla GFP.

5. The cell of claim 1, wherein said expression control sequences comprise a DNA damage induced promoter.

6. The cell of claim 5, wherein said promoter is GADD45a.

7. The cell of claim 1, wherein the cell is a eukaryotic cell.

8. The cell of claim 7, wherein the cell is a mammalian cell.

9. A cell line comprising at least one cell engineered to:
a) constitutively express a fluorescent protein in fusion with a centromere protein; and
b) inducibly express a nitroreductase (NTR) enzyme reporter gene under the control of a DNA damage induced promoter.

10. The cell line of claim 9, wherein said centromere protein is centromere protein A (CENP-A).

11. The cell line of claim 9, wherein, said DNA damage induced promoter is GADD45a.

12. The cell line of claim 9, wherein the fluorescent protein is a Green Fluorescent Protein (GFP) or a functional GFP analogue.

13. The cell line of claim 12, wherein the fluorescent protein is selected from wild type GFP, EGFP, EYFP, ECFP, F64L-GFP, BFP, NFP and Renilla GFP.

14. The cell line of claim 9, wherein the cell is a mammalian cell.

15. A process for producing a cell line including at least one cell comprising first and second reporter gene constructs capable of being expressed, which process comprises:
a) transfecting one or more cells with a first and a second recombinant expression vector, wherein:
i) said first recombinant expression vector comprises a first reporter gene construct comprising a nucleic acid molecule encoding a fluorescent protein fused to a centromere protein said nucleic acid sequence operably linked to and under the control of a promoter that is constitutively active; and
ii) said second recombinant expression vector comprises a second reporter gene construct comprising a nucleic acid molecule comprising one or more expression control sequences operably linked to a sequence encoding a nitroreductase (NTR) enzyme; and
b) selecting transfected cells which express at least one and optionally both first and second reporter genes.

16. The process of claim 15, wherein said at least one cell is transfected with said first recombinant expression vector followed by said second recombinant expression vector.

17. A method for determining the effect of a test agent on a phenotypic property of a cell, which method comprises:
a) providing cells comprising:
i) a first reporter gene construct comprising a nucleic acid molecule encoding a fluorescent protein capable of emitting fluorescent light of a first wavelength (λ₁) and being fused to a centromere protein said nucleic acid sequence operably linked to and under the control of a promoter that is constitutively active; and
ii) a second reporter gene construct comprising a nucleic acid molecule comprising one or more expression control sequences operably linked to a sequence encoding a nitroreductase (NTR) enzyme;
and wherein said cells are marked with a DNA staining dye that emits fluorescent light of a second wavelength (λ₂) when bound to DNA;
b) contacting a population of said cells with:
iii) a test agent or a control; and
iv) a substrate for nitroreductase wherein said substrate is capable of emitting fluorescent light of a third wavelength (λ₃);
under conditions permitting expression of said first and second reporter gene constructs; and
c) measuring the fluorescence intensity at different wavelengths of emitted light in said cell population;
wherein a difference in fluorescence intensity in cells treated with said test agent compared with cells treated with said control is indicative of the effect of said test agent on the phenotypic property of said cell.

18. The method of claim 17, wherein the fluorescent protein is a Green Fluorescent Protein (GFP) or a functional GFP analogue.

19. The method of claim 18, wherein the fluorescent protein is selected from wild type GFP, EGFP, EYFP, ECFP, F64L-GFP, BFP, NFP and Renilla GFP.

20. The method of claim 17, wherein said expression control sequences comprise a DNA damage induced promoter.

21. The method of claim 20, wherein said promoter is GADD45a.

22. The method of claim 17, wherein said nitroreductase substrate is a dye comprising at least one NO₂.

23. The method of claim 22, wherein said dye is a cyanine dye or a squaraine dye.

24. The method of claim 23, wherein said cyanine dye molecule is cell permeable.

25. The method of claim 22, wherein the dye has the formula: 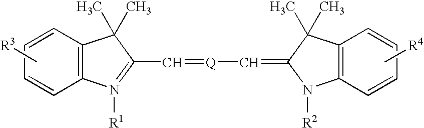
wherein:
Q is a fragment selected from the group consisting of: 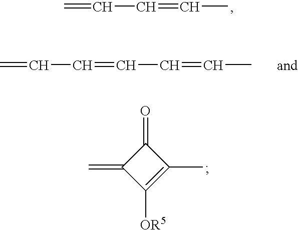
R¹ and R² are independently selected from C₁-C₄ alkyl, -(CH₂)ₙ—P,—{(CH₂)₂—O}ₚ—R⁶ and the group W; where P is selected from COOR⁷, SO₃⁻ and OH, W is mono- or di-substituted nitrobenzyl, R⁶ is methyl or ethyl, R⁷ is selected from H, C₁-C₄ alkyl and CH₂OC(O)R⁸, where R⁸ is methyl, or t-butyl, n is an integer from 1 to 10, and p is an integer from 1 to 3;
groups R³ and R⁴ are independently selected from hydrogen, NO₂, halogen, SO₃⁻, C₁-C₄ alkoxy and -(CH₂)ₘ—COOR⁷; where R⁷ is hereinbefore defined and m is 0 or an integer from 1 to 5;
R⁵ is hydrogen or C₁-C₄ alkyl;
provided that at least one of groups R¹, R², R³ and R⁴ comprises at least one NO₂ group.

26. The method of claim 25, wherein one of groups R¹ and R² is selected from group W where W is selected from: 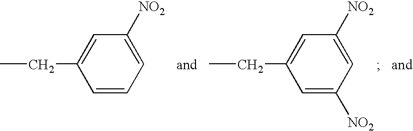
remaining R¹ or R² is selected from methyl, ethyl, the group -{(CH₂)₂—O}ₚ—R⁶ and the group -(CH₂)ₙ—COOR⁷, where R⁶ is methyl or ethyl, and R⁷ is H or C₁-C₄ alkyl.

27. The method of claim 25, wherein W is the group: 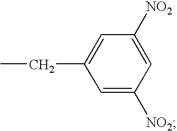
and remaining R¹ or R² is hereinbefore defined.

28. The method of claim 17, wherein the cell is a eukaryotic cell.

29. The method of claim 28, wherein the cell is a mammalian cell.

30. The method of claim 17, wherein said test agent is a chemical entity selected from a drug, a food dye, a hormone, a toxin, an alkylating agent, an oxidising agent, a carcinogen.

31. The method of claim 17, wherein said test agent is a physical agent selected from electromagnetic radiation (e.g. UV, X-ray, microwave), β⁻ radiation, heat.

32. The method of claim 17, wherein said phenotypic property is selected to be cell number, nuclear morphology, micronuclear frequency, centromere status of micronuclei and reporter gene expression.

33. A diagnostic kit for identifying and characterising a toxic agent, said kit comprising:
a) at least one cell comprising:
i) a first reporter gene construct comprising a nucleic acid molecule encoding a fluorescent protein fused to a centromere protein said nucleic acid sequence operably linked to and under the control of a promoter that is constitutively active; and
ii) a second reporter gene construct comprising a nucleic acid molecule comprising one or more expression control sequences operably linked to a sequence encoding a nitroreductase (NTR) enzyme; and
b) a nitroreductase (NTR) enzyme substrate.
